(19) United States
(12) Patent Application Publication
(10) Pub. No.: US 2015/0359637 A1
(43) Pub. Date: Dec. 17, 2015

(54) MICRO AND MACRO TEXTURING OF MACHINED OBJECTS

(71) Applicant: LABORATOIRES BODYCAD INC., Quebec (CA)

(72) Inventors: Florent MIQUEL, Quebec (CA); Jean ROBICHAUD, Quebec (CA)

(21) Appl. No.: 14/409,096

(22) PCT Filed: Jun. 26, 2013
(86) PCT No.: PCT/CA2013/000607

§ 371 Date: Dec. 18, 2014

Related U.S. Application Data
(60) Provisional application No. 61/664,381, filed on Jun. 26, 2012.

(51) Int. Cl. A61F 2/30; B23K 26/38; B23K 26/00

(52) U.S. Cl. CPC A61F 2/30; B23K 26/0084; B23K 26/385

(57) ABSTRACT

There is provided a method for texturing at least one object surface of a machined object for placement at an articular joint, the articular joint having a bodily fluid flowing therethrough. The method comprises at least one of machining a plurality of spaced macro-sized cavities on the at least one object surface, each one of the plurality of cavities configured to retain the bodily fluid therein, and machining a plurality of micro-sized ridges on the at least one object surface, each one of the plurality of ridges con figured to guide the bodily fluid on the at least one object surface.

### CROSS-REFERENCE TO RELATED APPLICATIONS

This patent application claims priority of U.S. provisional Application Ser. No. 61/664,381, filed on Jun. 26, 2012.

### TECHNICAL FIELD

The present invention relates to the field of computer-aided machining, in particular to methods of manufacturing a machined object such as a prosthesis having a textured surface.

### BACKGROUND OF THE ART

Success or failure of a prosthesis depends greatly on creating and maintaining an interface between the prosthesis and surrounding bone and tissue. Indeed, regardless of the implantation site, bone volume and quality, it is desirable for a prosthesis to present a surface which will not disrupt, yet may even enhance, the bone healing process.

When implanting a prosthesis in an articular region, it is desired that proper lubrication at the articular joint as well as reduced friction occur during movement. This in turn improves the functionality of the prosthesis while preventing subsequent wear or failure thereof. However, asperities on the surface of a prosthesis may prevent proper flow of bodily fluids.

There is therefore a need for a prosthesis having an improved textured surface.

### SUMMARY

There is described herein a method for improving lubrication and reducing friction at an articular joint where a machined object such as a prosthesis is implanted and adapted to mate with a bone surface or with another prosthesis. At least one surface of the machined object is textured to achieve at least one of a macro-sized topology and a micro-sized topology. The macro-sized topology comprises a plurality of cavities adapted to retain therein a bodily fluid flowing through a cavity of the articular joint. The micro-sized topology comprises a plurality of ridges adapted to guide a flow of the bodily fluid. Provision of the ridges further creates a plurality of asperities on the machined object surface for adjusting a contact area between the prosthesis surface and a mating surface.

In accordance with a first broad aspect, there is provided a method for texturing at least one object surface of a machined object for placement at an articular joint, the articular joint having a bodily fluid flowing therethrough. The method comprises at least one of machining a plurality of spaced macro-sized cavities on the at least one object surface, each one of the plurality of cavities configured to retain the bodily fluid therein, and machining a plurality of micro-sized ridges on the at least one object surface, each one of the plurality of ridges configured to guide the bodily fluid on the at least one object surface.

In accordance with a second broad aspect, there is provided a machined object for placement at an articular joint, the articular joint having a bodily fluid flowing therethrough. The machined object comprises a body having at least one object surface having machined thereon at least one of a plurality of spaced macro-sized cavities, each one of the plurality of cavities configured to retain the bodily fluid therein, and a plurality of micro-sized ridges, each one of the plurality of ridges configured to guide the bodily fluid on the at least one object surface.

In this specification, the term “machining” should be understood to mean any technique used to change the shape, surface finish, and/or mechanical properties of a material by the application of specials tools and equipment. This includes, but is not limited to, lapping, grinding, broaching, milling, nibbling, polishing, buffing, etching, and shearing using various cutting tools with blades and/or lasers.

### BRIEF DESCRIPTION OF THE DRAWINGS

Further features and advantages of the present invention will become apparent from the following detailed description, taken in combination with the appended drawings, in which:

FIG. 1a is a flowchart of a computer-aided method for manufacturing a patient-specific prosthesis in accordance with an illustrative embodiment of the present invention;

FIG. 1b is a flowchart of the step of manufacturing a prosthesis of FIG. 1a;

FIG. 2a is a front perspective view of a prosthesis in accordance with an illustrative embodiment of the present invention;

FIG. 2b is a side perspective view of the prosthesis of FIG. 2a;

FIG. 3a is a top view of a micro-textured external surface of the prosthesis of FIG. 2a;

FIG. 3b is a close-up view of the micro-textured external surface of FIG. 3a;

FIG. 4a is a top view of a macro-textured external surface of the prosthesis of FIG. 2a; and

FIG. 4b is a close-up view of the macro-textured external surface of FIG. 4a.

It will be noted that throughout the appended drawings, like features are identified by like reference numerals.

### DETAILED DESCRIPTION

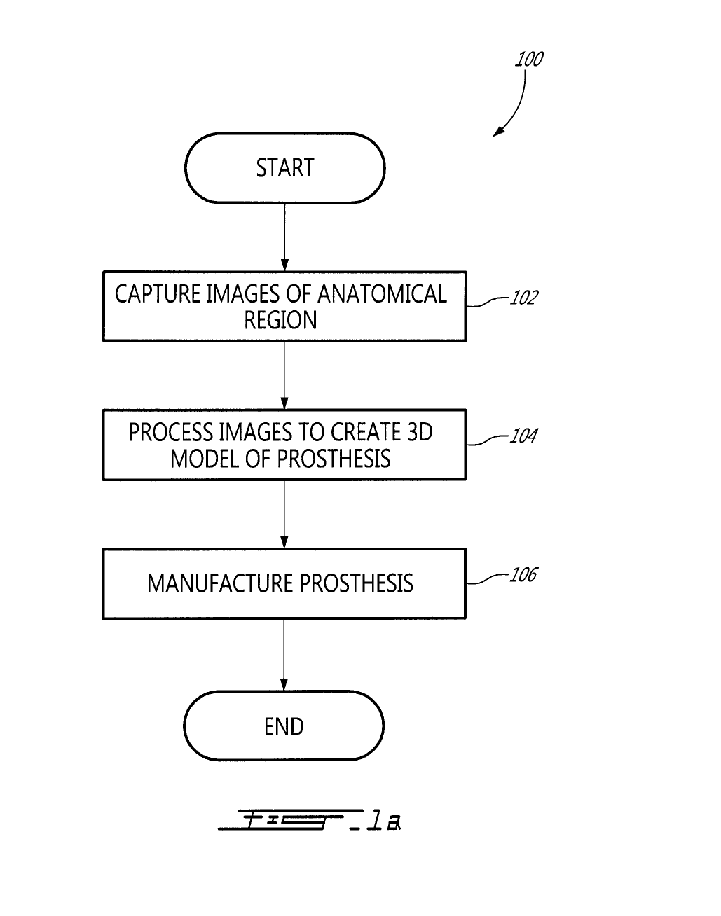

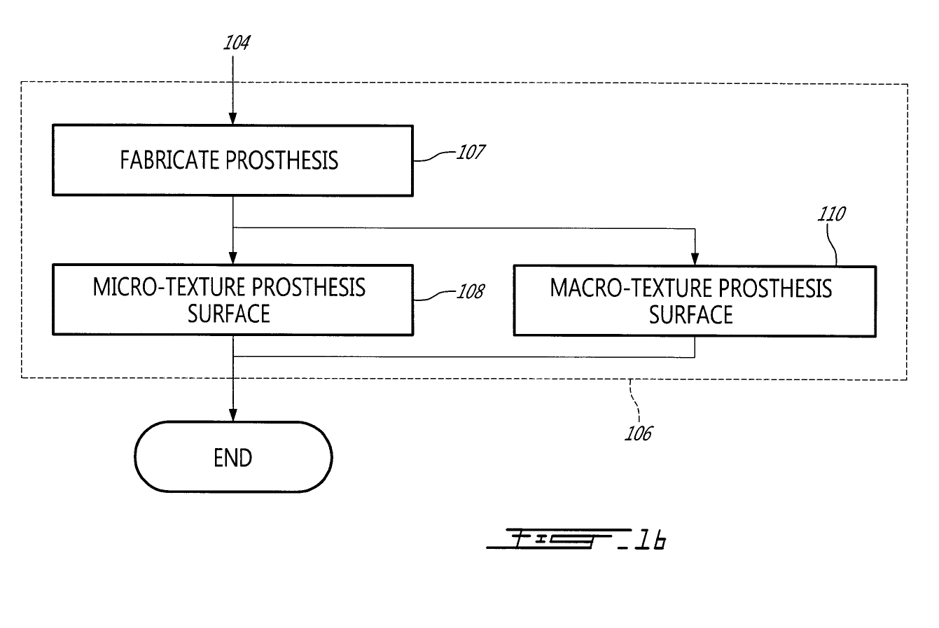

Referring to Figure la and FIG. 1b, a computer-aided method 100 for manufacturing a prosthesis will now be described. It should be understood that, although the description below refers to the manufacturing of a prosthesis, other components, whether they are made of metal, plastic, or any other suitable material, may also apply. In particular, any machined object which may be implanted at an articulation and is to interact with or be mated with the patient's anatomical structures and/or other machined components may apply.

The method comprises image capturing at step 102, which refers to acquiring image data of the patient's anatomical region where the prosthesis is to be implanted. Such anatomical region may for example comprise the hip, knee, and ankle regions when total knee replacement surgery is concerned. It should be understood that other anatomical regions, such as the mouth, ear, hand, etc., may be imaged in the process of manufacturing other types of prostheses. The images may be obtained from scans generated using Magnetic Resonance Imaging (MRI), Computed Tomography (CT), ultrasound, x-ray technology, optical coherence tomography, or the like.

Image capturing 102 may be done along one or more planes throughout the body part, such as sagittal, coronal, and transverse. In some embodiments, multiple orientations are performed and the data may be combined or merged during the processing phase (step 104). For example, a base set of images may be prepared on the basis of data acquired along a sagittal plane, with missing information being provided using data acquired along a coronal plane. Other combinations or techniques to optimize the use of data along more than one orientation will be readily understood by those skilled in the art. The captured images may further be provided in various known formats and using various known protocols, such as Digital Imaging and Communications in Medicine (DICOM), for handling, storing, printing, and transmitting information. Other exemplary formats are GE SIGNA Horizon LX, Siemens Magnatom Vision, SMIS MRD/SUR, and GE MR SIGNA 3/5 formats.

The images, once captured, may be processed using computer software to create a three dimensional (3D) model of the prosthesis (step 104), which is adapted to fit the patient's unique anatomical region, e.g. a damaged knee joint, for which the images have been captured. Using such a 3D model, it can be ensured that the prosthesis provides adequate integration with surrounding bone. Once the 3D model has been created, the prosthesis may be manufactured from a suitable material chosen for biocompatibility, such as a metal alloy, titanium, medical grade stainless steel, tantalum, composite, and ceramics, using computer-aided machining (CAM) for free-form machining thereof (step 106). In particular, micro-sized and macro-sized topologies may be machined on at least one of the external surface and the internal surface of the prosthesis. Step 106 may thus comprise at least one of micro-texturing (step 108) and macro-texturing the prosthesis surface (step 110) after a rough or initial fabrication of the prosthesis (step 107).

Response of tissues to a prosthesis is indeed largely controlled by the nature and texture of the surface of the prosthesis. Unlike smooth surfaces, textured prosthesis surfaces exhibit more surface area for integrating with the surrounding bone via osseo integration, which is a process in which osseous tissue attaches to an inert material without intervening connective tissue. Textured prosthesis surfaces further enable in growth of tissue. As such, texturing the surface of a prosthesis may enhance cellular activity and improve bone apposition, especially when bone volume and quality are poor. Moreover, texturing a prosthesis surface may improve lubrication of the articular surface, thus improving the functionality of the prosthesis in the implanted region.

Depending on the scale of the features provided on the prosthesis surface, surface roughness may be divided into macro- and micro-sized topologies. Macro-sized topologies with high surface roughness help in initial prosthesis stability and provide volumetric spaces for bone growth. Micro-sized topologies on the other hand enhance osteoconduction, i.e. in-migration of new bone, through changes in surface topology and osteoinduction, i.e. new bone differentiation, along the prosthesis surface by using the prosthesis as a vehicle for local delivery of bioactive agents. Macro-sized topologies illustratively have a surface roughness in the range of millimeters to microns whereas micro-sized topologies illustratively have a surface roughness in the range of microns.

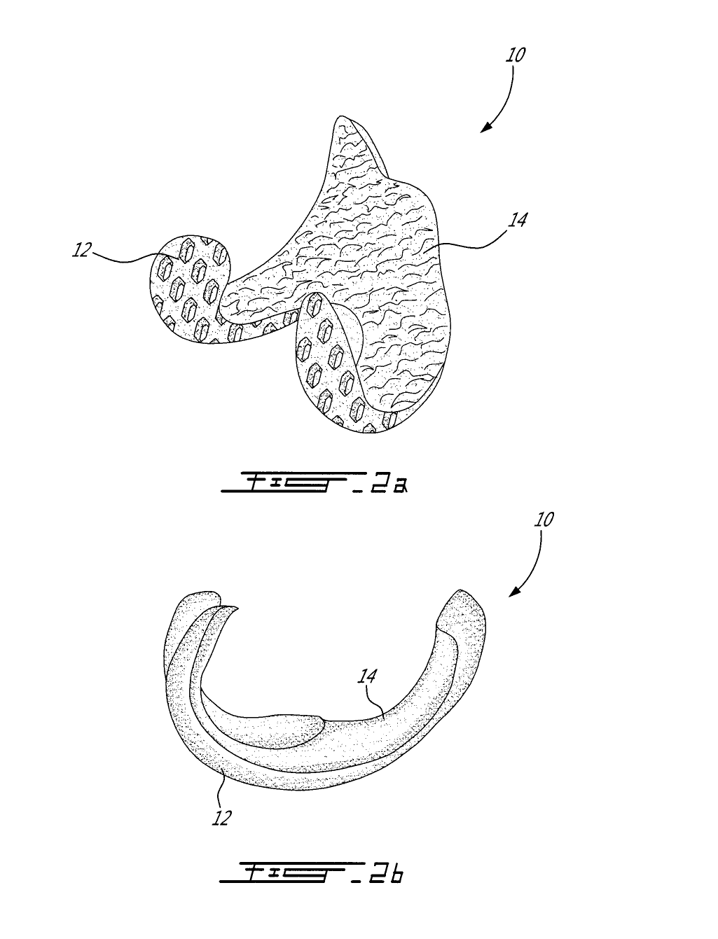

Referring to FIG. 2a and FIG. 2b, a prosthesis 10 having an external surface 12 and an internal surface 14, at least one of which may be textured, may result from implementation of the method 200. The prosthesis 10 is illustratively adapted to be positioned on a patient's femur (not shown) at a damaged knee joint for mating with a surface of the patient's tibia (not shown). Although a femoral prosthesis 10, and more particularly a femoral component, used in knee replacement procedures has been shown for illustrative purposes, it should be understood that tibial or patellar components may apply. It should also be understood that prostheses used for repairing articular joints, such as an elbow, shoulder, wrist, or hip, other than a knee may be used. Other types of prostheses known to those skilled in the art , such as facial or dental prostheses, may also apply.

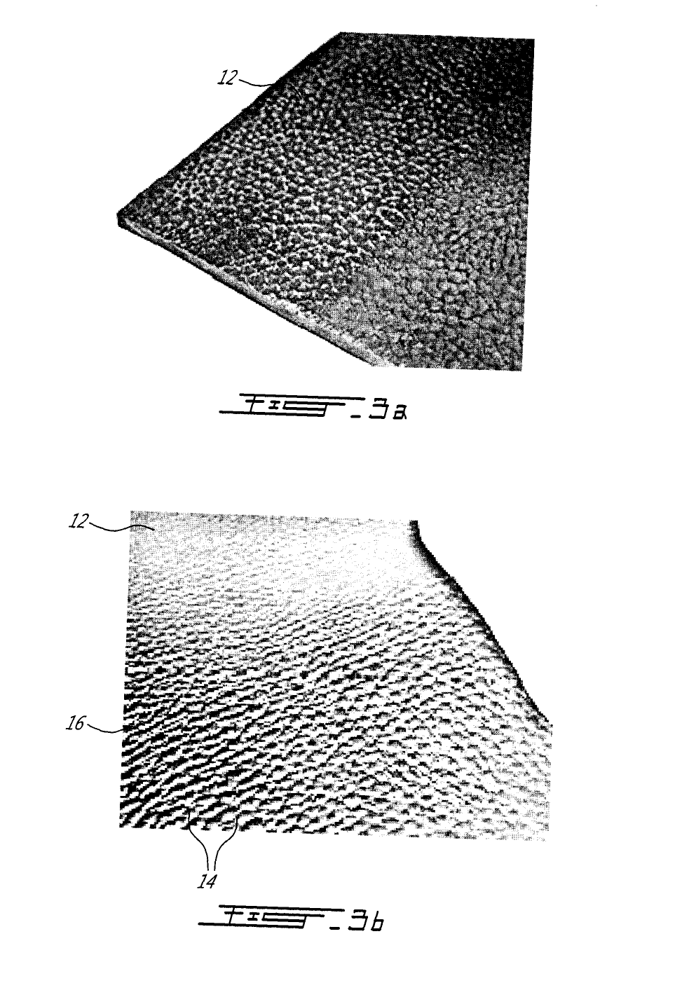

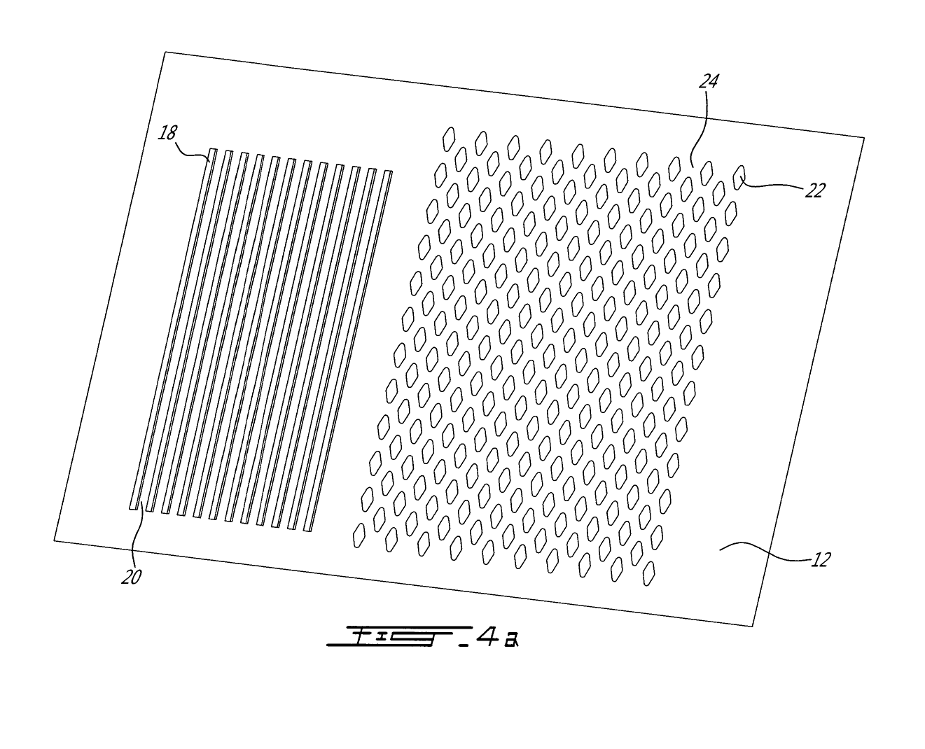

As seen in FIG. 3a and FIG. 3b, a surface of the prosthesis 10, such as the external surface 12, may be micro-textured. In this case, micro-machining may be achieved using a laser, such as an excimer laser, which alters the external surface 12 of the prosthesis 10 to create a pattern of ripples or ridges as in 14. The ridges 14 may be formed as creases in the surface 12 and may extend in various directions (not shown) along the surface 12 with paths of adjacent ridges 14 crossing. As a result, the surface 12 may exhibit a number of asperities 16 delimited by adjacent ridges 14 and disposed at a higher elevation than the latter. The surface 12 may then exhibit a generally ridged or otherwise wrinkled texture. Laser treatment enables treatment of the surface 12 of the prosthesis 10 without direct contact with the latter in addition to providing better control on the topology of the surface. For example, using such a treatment, it may be possible to control the thickness of the ridges 14 as well as the depth of the ridges 14 relative to the surface 12. Illustratively, micro-sized topologies having at least one dimension (e.g. height and/or width) in the order of less than 0.9 mm, and more particularly in the range between 100 and 300 microns may be achieved. In one exemplary embodiment, the range is between 100 and 900 microns. In another exemplary embodiment, the range is between 300 and 500 microns. Other ranges will be readily apparent to those skilled in the art. Also, machining techniques other than lasers, for example scraping using a thin and sharp spike made of a hard material, such as diamond or sapphire, may be used to achieve a micro-textured surface.

Varying the positioning and/or spacing of the ridges 14, and accordingly of the asperities 16, on the surface 12 enables control of the contact area between the prosthesis 10 and the surrounding bone at the articular region where the prosthesis 10 is implanted. Indeed, when the surface 12 is mated with another mating surface, such as the surface of a bone (not shown) of the articular joint, the asperities 16, being provided at a higher elevation than the ridges 14, make contact with the mating surface while the ridges 14 are spaced from the mating surface. Thus, by providing more or less asperities 16, it may be possible to control the level of contact or adhesion of the prosthesis surface 12 to the mating surface, e.g. the surface of the patient's tibia. It will therefore be understood that the design of the pattern of ridges 14 may depend on the type of prosthesis 10 to be implanted as well as on the bone surface the prosthesis 10 is to be in contact with. It will also be understood that the mating surface may be the surface of another machined object, e.g. a prosthesis component, placed at the articular joint.

In addition, micro-texturing the surface 12 may allow guiding a flow of a bodily fluid, such as a synovial fluid reducing friction at articular joints during movement, on the surface 12. In particular, the bodily fluid may flow through the micro-sized features, e.g. the ridges 14, such that the flow of the bodily fluid on the surface 12 is guided by the ridges 14 in accordance with the arrangement thereof. This in turn ensures that the prosthesis 12 may function as close as possible to the manner in which the damaged or missing body part the prosthesis 12 replaces used to function. The micro-textured surface 12 may also enable use of the prosthesis 10 for local delivery of bioactive agents. For instance, Bone Morphogenetic Proteins (BMPs) may be delivered using the ridges 14 formed on the micro-textured surface 12 for inducing the formation of bone and cartilage around the prosthesis 10. In this manner, integration over time of the prosthesis 10 with surrounding bone and tissue may be promoted, thus reducing the probability of early wear and failure of the prosthesis 10.

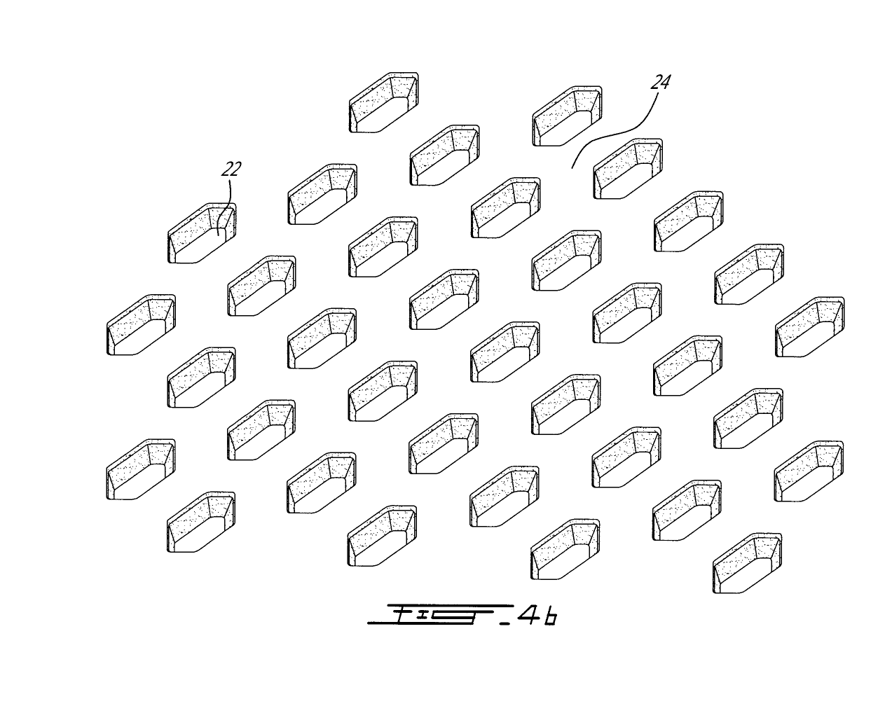

Referring to FIG. 4a and FIG. 4b, a surface of the prosthesis 10, such as the external surface 12, may further be macro-textured. The macro texture is illustratively obtained by removing material at discrete locations on the surface 12. Alternatively, the surface 12 may be deformed to achieve a similar effect. For this purpose, mechanical machining may be used to texture the surface 12 by means of physical forces. Such machining may for example be achieved using a milling cutter of a shape and size adapted to accurately produce the desired surface topology. Lapping, grinding, broaching, milling, nibbling, polishing, buffing, etching, and shearing using various cutting tools with blades and/or lasers may also apply. In this manner, macro-sized topologies in the order of 300 microns to 1 mm may be obtained. Other dimensions consistent with the intended purpose of the macro-sized surface topology can be achieved. Still, it should be understood that the macro texture may also be obtained by adding material to the surface 12.

The macro-textured surface 12 illustratively comprises a pattern of cavities or depressions, as in 18 or 22, having a size in the range of millimeters to microns in at least one dimension and which may be generated using computer software. In one embodiment, the pattern of cavities 18, 22 is regular. It should however be understood that an irregular pattern may be achieved. Also, the cavities 18, 22 are illustratively not interconnected although it should be understood that in some embodiments, some or all of the cavities 18, 22 may be interconnected such that an unobstructed passageway is formed therebetween. The positioning (e.g. spacing, orientation), number, size, and shape of the depressions may depend on the geometry of the prosthesis 10, on the material used to manufacture the latter, on the geometry of the articular joint onto which the prosthesis 10 is to be affixed, as well as on the viscosity and texture of the friction fluid at the articular joint. For instance, the surface 12 may comprise a plurality of substantially parallel elongated channels 18 separated by elongated spacings 20. In one embodiment, equal spacings 30 are provided to achieve a regular pattern.

The surface 12 may also comprise a pattern of spaced dents 22 each shaped as a diamond or any other suitable shape, such as an annular, triangular, or rectangular shape. In one embodiment, the diamond-shaped dents 22 are arranged so as to be aligned in equally-spaced rows (not shown) to achieve a regular pattern. Other configurations may apply. The machined channels 18 and dents 22 may be disposed at a lower elevation than that of the surrounding surface 12 of the prosthesis 10. In this manner, bodily fluid present at the articular joint and flowing on the surface 12 may be captured within each one of the channels 18 and/or dents 22. Thus, the channels 18 and dents 22 enable retention of drops of bodily fluids therein, in turn improving lubrication at the articular joint. The macro-sized topology of the surface 12 may further prevent scar tissue from laying down uniformly on the surface 12, thus preventing complications, such as capsular contracture due to a tightening of the scar tissue.

It should be understood that both macro-sized and micro-sized topologies may be machined on the same prosthesis surface, such as the external surface 12, in such a manner that they do not directly overlap. Also, although texturing of the prosthesis surface has been described herein with reference to the external surface 12, it should be understood that the internal surface 14 of the prosthesis 10 may also be textured to improve lubrication where the prosthesis is to be implanted and thus reduce friction at the articular joint during movement. At least one of the external surface 12 and the internal surface 14 may therefore be textured.

It should be noted that the embodiments of the invention described above are intended to be exemplary only. The scope of the invention is therefore intended to be limited solely by the scope of the appended claims.

1. A method for texturing at least one object surface of a machined object for placement at an articular joint, the articular joint having a bodily fluid flowing therethrough, the method comprising:
at least one of
machining a plurality of spaced macro-sized cavities on the at least one object surface, each one of the plurality of cavities configured to retain the bodily fluid therein; and
machining a plurality of micro-sized ridges on the at least one object surface, each one of the plurality of ridges configured to guide the bodily fluid on the at least one object surface.

2. The method of claim 1, wherein machining the plurality of micro-sized ridges comprises machining the plurality of micro-sized ridges so as to define on the at least one object surface a pattern of asperities between adjacent ones of the micro-sized ridges, the asperities having an elevation greater than that of the plurality of micro-sized ridges.

3. The method of claim 2, wherein the at least one object surface is configured to mate with a mating surface provided at the articular joint, with the asperities making contact with the mating surface and the micro-sized ridges spaced from the mating surface, and wherein machining the plurality of micro-sized ridges comprises adjusting at least one of a positioning and a spacing of the ridges for controlling the pattern of the asperities, thereby controlling a level of contact of the at least one object surface with the mating surface.

4. The method of claim 1, wherein machining the plurality of micro-sized ridges comprises machining the plurality of micro-sized ridges each having a dimension lower than 0.9 mm.

5. The method of claim 1, wherein machining the plurality of micro-sized ridges comprises using a laser to create the plurality of micro-sized ridges.

6. The method of claim 1, wherein machining the plurality of spaced macro-sized cavities comprises removing material from a plurality of discrete locations on the at least one object surface.

7. The method of claim 1, wherein machining the plurality of spaced macro-sized cavities comprises using a laser to create the plurality of spaced macro-sized cavities.

8. The method of claim 1, wherein machining the plurality of spaced macro-sized cavities comprises applying a physical force to deform the at least one object surface at a plurality of discrete locations thereon.

9. The method of claim 1, wherein machining the plurality of spaced macro-sized cavities comprises machining in the at least one object surface a plurality of substantially parallel channels, each one of the plurality of channels disposed at a lower elevation than that of the at least one object surface and adapted to retain therein one or more drops of the bodily fluid flowing on the at least one object surface when the machined object is placed at the articular joint.

10. The method of claim 1, wherein machining the plurality of spaced macro-sized cavities comprises machining the plurality of macro-sized cavities each having a dimension in a range between 300 microns and 1 millimeter.

11. A machined object for placement at an articular joint, the articular joint having a bodily fluid flowing therethrough, the machined object comprising:
a body having at least one object surface having machined thereon at least one of
a plurality of spaced macro-sized cavities, each one of the plurality of cavities configured to retain the bodily fluid therein; and
a plurality of micro-sized ridges, each one of the plurality of ridges configured to guide the bodily fluid on the at least one object surface.

12. The machined object of claim 11, wherein the at least one object surface comprises at least one of an internal surface and an external surface of the body.

13. The machined object of claim 11, wherein a pattern of asperities is defined between adjacent ones of the plurality of micro-sized ridges, the asperities having an elevation greater than that of the plurality of micro-sized ridges.

14. The machined object of claim 13, wherein the at least one object surface is configured to mate with a mating surface provided at the articular joint, with the asperities making contact with the mating surface and the micro-sized ridges spaced from the mating surface, at least one of a positioning and a spacing of the ridges selected for controlling the pattern of the asperities, thereby controlling a level of contact of the at least one object surface with the mating surface.

15. The machined object of claim 11, wherein each one of the plurality of micro-sized ridges has a dimension lower than 0.9 mm.

16. The machined object of claim 11, wherein the plurality of spaced macro-sized cavities comprises a plurality of substantially parallel channels each disposed at a lower elevation than that of the at least one object surface and adapted to retain therein one or more drops of the bodily fluid flowing on the at least one object surface when the machined object is placed at the articular joint.

17. The machined object of claim 11, wherein each one of the plurality of spaced macro-sized cavities has a dimension in a range between 300 microns and 1 millimeter.
